(19) United States
(12) Patent Application Publication
(10) Pub. No.: US 2022/0219842 A1
(43) Pub. Date: Jul. 14, 2022

(54) CLOSED CONVENIENCE KITS FOR STERILIZED MEDICINE PREPARATION

(71) Applicant: Thorne Intellectual Property Holdings, LLC, Bountiful, UT (US)

(72) Inventors: Matthew Stephen Ward, Orem, UT (US); Gale Harrison Thorne, Bountiful, UT (US); Gale Harrison Thorne, JR., Bontiful, UT (US)

(73) Assignee: Thorne Intellectual Property Holdings, LLC, Bountiful, UT (US)

(21) Appl. No.: 17/300,842

(22) Filed: Dec. 13, 2021

Related U.S. Application Data
(63) Continuation-in-part of application No. 16/783,780, filed on Feb. 6, 2020, now Pat. No. 11,345,046.

(51) Int. Cl. B65B 3/00; A61M 5/00; B65B 55/02; A61B 50/33

(52) U.S. Cl. CPC B65B 3/003; A61M 5/002; A61B 50/33; B65B 55/027; A61M 5/008

(57) ABSTRACT

Methods for providing a plurality of convenience kits for sterilizing and delivering a quantity of medicine into the safety of a sterile chamber inside closed system (which can be disposed in a potentially contaminating field environment) are disclosed for container filling applications including Avastin. fortified antibiotic eye drops, mitomycin and hazardous drugs, in general. The containers can include medical syringes, eye drop bottles, vials and product quality assurance containers. A syringe needle cap effective in containment of fluids dispensed from a syringe while being primed is also disclosed.

### CONTINUATION-IN-PART

This Patent Application is a CONTINUATION-IN-PART of U.S. patent application Ser. No. 16/873,780, titled BAGGED BOTTLE FILLING AND CAPPING DEVICES AND METHODS submitted by Gale H. Thorne, Jr., et al., and contains information related to U.S. Pat. Nos. 10,555,872 B2, 10,800,556 B2 and 10,940,087 B2 but includes methods, applications and breadth of scope, unforeseen and not disclosed therein by the inventors.

### FIELD OF INVENTION

Inventions disclosed herein relate generally to convenience kits and associated applications for medical procedures involving steps for mixing, sterilizing, transferring and capping medicine containers which require sterile conditions in processing, storage and pre-use. Of particular note, the convenience kits and methods of use are applied to steps which can occur in a potentially contaminating environment and still produce a sterile product. Therefore, the field of invention is particularly related to sterile kits (disposed within convenience kits) and to methods which employ preassembled parts provided sterilized within the protective enclosure of a closed and sealed apparatus having only a pathway or pathways into the apparatus through sterilizing assemblies which are primarily used for introducing medical product therein, thereby providing a protectively packaged sterile product without requiring employment of a laminar flow hood or other sterilization assurance level (SAL) product manipulating devices. Each convenience kit is a single use tool which is specifically designed and assembled to be used in preparation of a particular medicine.

### BACKGROUND AND DESCRIPTION OF RELATED ART

U.S. Pat. No. 10,555,872 B1 (patent 872) discloses and claims a convenience kit for sterilizing and delivering liquids into the safety of a. sterile environment inside a plastic bag (which can be disposed in a potentially contaminating field environment) wherein a sterilized liquid is dispensed into a bottle which is capped and sealed before removal from the bag. The patent 872 convenience kit can be provided in a solitary format or, as a subkit, combined with other associated items in a more inclusive convenience kit. In short, convenience kits made, fin example, according to U.S. Pat. No. 10,555,872 B1, provide opportunity for accomplishing an aseptic liquid sterilizing transfer, a task which is commonly associated with capability of a laminar flow hood or other similar equipment, in field environments and other areas which are remote from facilities having such equipment,

Because kit items made and used according to the present inventive sterile kit cannot be removed or replaced without affecting sterility, a complete set of items required for sterile kit performance for a specific sterilizing and filling application must be provided as a closed system which is sealed and sterilized before use. Because particular kit applications are kit specific and have specific application and item requirements, methods and apparatus used for such applications often require novelty in selection and method of use, resulting in a special kit with novel features and uses being specifically designed for each such different application.

Thus, novel items disclosed and claimed hereafter provide means for performing unique kit single use functions which deviate and are not obvious from disclosures in prior related patents, published patent applications and other methods and apparatus in the public domain, while providing for quality kit prepared products using unique combinations of item design and selection for meeting each specific product application requirement.

As background mixing and compounding of medications, in a pharmacy or other medical facility, conventional techniques involve use of laminar flow hoods and other “closed” systems and strict aseptic technique and facilities to maintain sterility. Because of such, the United States Pharmacopeia Convention (USP) has found it necessary to establish standards, currently published in a Chapter titled (797) PHARMACEUTICAL COMPOUNDING—STERILE PREPARATIONS (referenced hereafter as Chapter 797), being a latest revised version dated 2021. The term “closed” has been italicized because equipment used is, in reality, open to the exterior environment, but employ various controls such as laminar flow to create as closed an environment as possible. Such systems permit a high variety of sterile preparation to occur within the same environment, but each substance or device to be manipulated within the sterile field must be introduced in a manner requiring special training, standards and precautions to assure maintenance of product sterility in multi-use systems. This requirement establishes the basis for a significant portion of the current, revised Chapter 797. In the second paragraph of the INTRODUCTION of Chapter 797, the following comment is made concerning touch contamination:
Despite the extensive attention in this chapter to the provision, maintenance, and evaluation of air quality, the avoidance of direct or physical contact contamination is paramount. It is generally acknowledged that direct or physical contact of critical sites of CSPs with contaminants, especially microbial sources, poses the greatest probability of risk to patients. Therefore, compounding personnel must be meticulously conscientious in precluding contact contamination of CSPs both within and outside ISO Class 5 (see Table 1) areas.

It is well understood that standards for sterility and safety as disclosed in Chapter 797 are, at least, minimum and well worth abiding. However, providing a sterilizing pathway into the environment of a sterilized bag or other closed system containing pre-assembled and pre-sterilized components which can be filled with sterile product and manipulated external to the sterile environment, almost entirely, eliminates the possibility of contact contamination. As such, such a device renders moot many of the precautions recommended by 797 for manipulation of sterile product within the quasi-closed environment of a flow hood.

First, it should be recognized that the bag or container used for this invention and items therein represent a truly closed and continuously sterile system, because only matter sterilized by filtering can be delivered into the container permitting container contents to maintain a predetermined SAL. Such apparatus as this type of a closed system varies significantly from a quasi-closed system where sterile processing must be performed by highly skilled and extensively trained professionals using special techniques in scrupulously clean environments in order to maintain a desired sterilization assurance level (SAL). Unless the above disclosed closed container of the instant invention is damaged, concerns pertaining to the following items of Chapter 797 are significantly reduced or of no consequence:
1. Garbing to maintain product sterility;
2. Training for proper use of laminar flow hoods and other like equipment which is not required by a sterile bag or other truly closed and sterilized system;
3. Protecting and sampling airborne contaminates; and
4. Monitoring and cleaning surface contaminates surrounding the production site. Of course, items used within the bags should not be pyrogenic.
   Thus, it is considered a major object to define apparatus and methods for making and using truly closed and sterile kits into which all fluid, which is dispensed therein, is sterilized to preserve a desired in-container SAL which is an inventive aim of apparatus and methods disclosed hereafter.

Convenience kits have become commonly used appliances in medicine for a number of reasons. First, each convenience kit is usually made for a specific application. Contents, of each such kits, are prepared and provided in a form which most often reduces procedure steps and improves efficiency. Second, such kits can provide effective safety such as the kit disclosed in U.S. Pat. No. 9,449,521, titled METHODS FOR MAKING AND USING A VIAL SHIELDING CONVENIENCE KIT, issued May 28, 2013, which proved effective in providing additional safety to technicians and patients by keeping hazardous drug liquid and fumes fully contained.

For reasons cited supra and because of Chapter 797's allowance for mixing and delivery of medical product within a short time limit indicates acceptance of all facets of product production except for maintaining a desired SAL (underlining for emphasis), preparing sterile product in pre-sterilized sealed bags or containers provides a novel opportunity for medical product preparation safety. Whereas aseptic production of CSPs using current methods depends on the training and technique of the personnel involved, the novel system presented here provides improved safety as aseptic production of CSPs is completely independent of the training or technique of the user. As such, garbing, training, facility management, cleaning and monitoring issues, as exemplified by USP Chapter 797 should be reconsidered for applications employing sterile kits made in accordance with the instant invention and provided as disclosed and referenced herein.

### Terms and Definitions

Following is a list of terms and associated definitions which may be used herein, being provided for clarity and understanding, if and when used, to better disclose precepts of the instant invention:

### Chapter <797> Terms and Definitions:

CSP (abbreviation) for: Compounded Sterile Preparation, a preparation intended to be sterile by combining, admixing, diluting, pooling, reconstituting, repackaging, or otherwise altering a drug product or bulk drug substance.
Category 1 CSP: A CSP that is assigned a BUD (Beyond Use Date) of 12 hours or less at controlled room temperature or 24 hours or less refrigerated that is compounded in accordance with all requirements for Category 1 CSPs in Chapter <797>.
Category 2 CSP: A CSP that is assigned a BUD (Beyond Use Date) of greater than 12 hours at controlled room temperature or greater than 24 hours, refrigerated that is compounded in accordance with all requirements for Category 2 CSPs in Chapter <797>.
Immediate Use CSP: A CSP compounded for use within a four hour period and not subject to the requirements of Category 1 or Category 2 CSPs. (Must use aseptic processes performed in accordance with evidence based information, not involving more than 3 different products, discarding unused product, and appropriate labeling.)

### Other Terms and Definitions:

at least one, n: one or more
apportioning device, n: a fluid dispensing instrument from which a measured quantity can be dispensed
bottle, n: a container for holding a quantity of medicine
chamber, n: an enclosed sterile space or cavity by which, according to the instant invention, liquid is displaced into containers, then capped within the sterile environment to prepare a sterile medicine for use outside the chamber.
convenience kit, n: (according to the instant invention) a self-contained, single-use convenience kit designed and assembled for preparing a specific medical product, each such kit comprising a sterile kit, a preparation (“prep”) kit providing any supporting items required exterior to the sterile kit, other parts associated with transport, instruction and packaging and waste disposal.
manifold connection, n, an interconnected channeling comprising four orthogonally disposed pathways with luer fittings affixed to each pathway
dead space, n: a volume of inaccessible fluid which is retained within a device after a procedure
digital, adj: relating to, or performance with thumb or fingers
ETO, n: acronym for ethylene oxide, a powerful sterilizing agent
field of use, n: a location in an uncontrolled environment in which potentially health-hazardous materials are present.
filter, n: a fluid transferring product using filtering material having a sufficiently small porous matrix to impede passage there through of a particulate of predetermined size; a medical grade sterilizing filter generally has a 0.2 micron pore size.
filter assembly, n: (a.k.a a sterilizing assembly) an apparatus which comprises a filter component whereby one of the opposing fittings is disposed for communicating fluid into a chamber which is closed and sterilized to a predetermined SAL, the filter assembly providing throughput for a fluid which is displaced therethrough into the chamber to ultimately fill a container with sterilized liquid. If the chamber is the inside of a bag, the communication pathway is via a hole in the bag, the hole being closed and sealed about the hole; if otherwise, the communication pathway is via fittings providing fluid flow into a chamber formed by the fittings and therefrom into receiving containers.
filter component, n: a component and housing having a pair of opposing fittings providing communicating conduits to and from a filter whereby a fluid is sterilized to a predetermined SAL when displaced therethrough.
fitting, n: a medical connector
formulate, v: to prepare a substance for use; when used for medicine preparation, it is understood preparation is according to a specific prescription
free plunger, n: a syringe plunger which is unattached to a plunger rod while the syringe is filled
insulated wrap, n: a flexible container which may be a bag or folded shield which is refused to provide a container in which enclosed parts can be maintained at a reduced temperature
interface gasket, n: an elongated hollow tube that is sized, shaped and disposed to be affixed along a filter component or other component conduit about a hole in a plastic bag and thereby provide a fluid tight seal about the hole in the bag
kit, n: a group of parts, provided within a single package for a particular, designated use
laminar flow hood, n: (a fume hood) a work-place enclosure in which micro-filtered air flow is directed to preclude, to a predetermined extent, contamination of sterile materials by airborne organisms
luer fitting, n: a medical connector having a frustoconically shaped connecting geometry which is in common use in medical practice
luer lock fitting, n: a luer fitting having a locking mechanism whereby a male and a female connector are securely, but releasably affixed one to the other
noninferior, adj: equal to or better than the object at hand
plastic bag, n: a sturdy container made of clear, pliant, sterilizable material which is sealed to provide a totally enclosed product shroud after displacing devices for use therein, the material being sufficiently pliant to permit digital product handling from outside the container
plate, n: a planar sheet of material comprising a pattern of holes in which caps are stored for capping containers, such as eye drop bottles
port or portal, n: an orifice site where through fluid is communicated (generally associated with a sealed conduit disposed there through)
prep kit, n: a part of a convenience kit, according to the present invention, which comprises items required exterior to a sterile kit for preparing a medical product
radiation, n: generally, gamma radiation imposed with sufficient intensity and time to sterilize a product to a desired SAL (sterility assurance level)
sterile chamber n: space within a sterile kit which is physically enshrouded, protected and maintained at a sufficiently low SAL to assure displacement of preparations there through, without undue contamination, the only fluid pathway for fluid entering into the chamber being a sterilizing device (e.g. a filter) by which all such fluid is sterilized to a predetermined SAL
sterile kit n: a separate kit which is part of a convenience kit prepared according to the present invention, the sterile kit comprising a closed chamber which is pre-sterilized, along with all devices provided therein and affixed thereto, to a predetermined SAL, and providing at least one pathway or into the chamber via sterilizing devices, such as a medical grade sterilizing filter; as an example, the closed chamber may be a plastic bag or a fluid pathway through a manifold.
tray, n: a convenience kit container, comprising a pattern of holes sized and shaped to securely hold liquid receiving vessels through filling, capping, and transporting
unitized, adj: a plurality of parts permanently joined to be handled and used as a single unit

### BRIEF SUMMARY AND OBJECTS OF THE INVENTIONS

In brief summary, inventions disclosed herein broaden the scope of uses for convenience kits for sterilizing, capping and filling in manners not disclosed by related art by providing different, non-obvious and new uses for convenience kits related to mixing, dispensing and providing an enclosed sterile space (a sterile chamber) in which a sterile liquid medicine is prepared for use outside each kit in what may be a potentially contaminating field environment.

Currently, medical preparations that must be delivered sterile are produced under guidance of USP Chapter 797 within the confines of a fume or laminar flow hood in a pharmacy or laboratory facility. As such, current practice prohibits preparation and delivery of sterile products at any site remote from such Chapter 797 compliant facilities, greatly limiting access to such important sterile preparations to many medical professionals and their patients where such advanced facilities do not exist or are inadequate to fill the scope of need. It is for the purpose of fulfilling this need that the instant inventions comprising the methods and single-use convenience kit assemblies are disclosed and claimed herein. A summary comparison of sterile kit compliance with 797 standards is provided in Table 1 below:

TABLE 1
Comparison of USP 797 Standards and Sterile Kit
manufacture and use standards

| | | Sterile Kit Environment, |
|---|---|---|
| | USP Standard | according to present |
| Description | Environment | invention |
| Filtration into | HEPA Filters | Syringe filters (0.2 micron) for |
| environment | (0.3 microns) | entry into a sterile chamber |
| Anteroom | Required | All Sterile Kits are manufactured |
| | | using an anteroom and |
| | | buffer room inside a certified |
| | | clean room environment as |
| | | required to minimize particulates. |
| | | HEPA filters are used |
| | | in these clean rooms with air |
| | | flow controls. Regularly |
| | | scheduled testing is performed |
| | | to certify clean room |
| | | standards. |
| Buffer Room | Required | All Sterile Kits are assembled |
| | | inside a certified buffer |
| | | room as required to meet GMP |
| | | standards for medical |
| | | devices. |
| Garbing | Required for all | Garbing requirements are met |
| | clean room | during manufacture of the |
| | operations | Sterile Kit. Use of the kit does |
| | | not require garbing since |
| | | all fluids must pass through |
| | | a sterile filter into the filling |
| | | environment of a sterile chamber. |
| Terminal | Drug/admixture | Sterilized bag or other sterilized |
| Sterilization | dependent. | container associated with |
| | Not all drugs can | the instant invention is sterilized |
| | be terminally | following kit assembly, |
| | sterilized. | All fluids are terminally sterilized |
| | | upon injection through |
| | | the .2 micron filter and |
| | | delivery to a sterile filling |
| | | chamber. |
| Sterility testing | Sampling | Sterile Kit sterilization is |
| | method—cannot | confirmed in each |
| | test | bag or device |
| | all finished | using a marker. GMP practices |
| | admixtures. | assures product integrity |
| | Sterility of all | including low particulate |
| | compounded | requirements during assembly |
| | solutions is based | in a certified clean room. All |
| | on statistical | fluids delivered through the |
| | analysis. | filter assembly are filtered |
| | | through .2 micron filters. |
| Environmental | Defined in | As described supra, clean |
| testing | USP 797 | rooms used for Sterile Kit |
| | documents, | assembly meet all environmental |
| | including but not | testing requirements |
| | limited to: | associated with clean room |
| | Glove testing | assembly. Compounding |
| | Air flow | steps during use of Sterile |
| | Surfaces | Kits involve sterile technique- |
| | | independent processing. |
| | | A sterile, filtered environment |
| | | during manufacture eliminates |
| | | bacteria and particulates |
| | | greater than .2 microns |
| | | within the sterile chamber. |
| | | Disposable, single use kits |
| | | provide a new “facility” and |
| | | new sterile “environment” |
| | | for each compounding |
| | | process. |
| Regular | Required and | Each sterile chamber provides |
| environment | technique | a new, clean environment |
| cleaning | dependent for | that is guaranteed sterile |
| | environment | |
| | sterility | |
| Environment | Meets USP | Meets and exceeds USP |
| comparison | Chapter 797 | Chapter 797 environment |
| Summary | environment | requirements |
| | requirements | |

In summary, sterile kits made according to the present invention, meet or exceed all required USP 797 requirements for a sterile admixing environment. In effect, the sterile kit creates (via a sterile chamber) a smaller version of a clean room where all activities relative to compounding are completed within the “facility” and “environment” of the fluid filling system.

Thus, it is a major object of invention disclosed herein to provide examples of alternate ways and means for achieving the intent and purpose of USP Chapter 797 by ways not currently possible or available with contemporary convenience kits or other modes of production.

The core item of each inventive kit made according to basic inventive properties of kits for sterilizing, filling and capping under strictly sterile conditions is a sterile kit having a sterile chamber comprising either product preparation within a closed pre-sterilized bag assembly or other closed system wherein containers may be filled and capped or within a strictly closed pre-sterilized interior pathway formed by conventional medical devices and fittings for fluid delivery which is securely affixed in fluid tight relationship with recipient closed containers. If such an item is a bag, the bag should be made from material that is not only pliant, but also sufficiently thick and hardy to permit digital interfacing without breaching, transparent to permit visual feedback and sealable to assure maintenance of sterility until purposely opened for access to capped and sealed preparations. Strictly closed systems should be provided by proved bags, connectors, tubes and other commercial devices which have a long and successful history of being able to be sterilized and maintain sterility when used properly:

Generally, within the scope of each of the instant inventions, there is at least one fluid pathway into each sterile chamber through a sterilizing assembly whereby fluid is sterilized to a desired SAL by flowing therethrough. If there are multiple fluid pathways, each pathway is provided with a sterilizing input assembly.

Prior related art has disclosed methods and apparatus for achieving objectives of sterilizing, filling and capping bottles, such as eye drop bottles, which are filled by dispensing liquid through open, superiorly disposed orifices which, for example, are used for autologous serum eye drops. However, the need and opportunity for applying other novel forms of the closed sterilization and filling technology is much broader, as exemplified by the breadth and comprehensiveness of USP Chapter 797. It is, therefore, a major object to provide novel methods and apparatus for satisfying needs for broadening use and, yet, strictly meeting (being “noninferior” in meeting) sterility requirements in medical product genesis.

In all cases, each convenience kit, made according to the instant invention, will be provided with a sterile kit having a sterile chamber in some form. Each such kit will only be used just once. Other items within the convenience kit, but separate from the sterile kit, are provided as needed to meet auxiliary medication, transport, disposal and instruction requirements. In all cases, where bags form the truly closed system, consideration should be given to facilitating in-bag manipulations from the bag exterior. Novel apparatus and methods for various kinds of medicine preparation are provided in the following examples.

A tray is disclosed in the cited prior art which employs bottle holding wells which are closed at the bottom. It has been found, in some cases, that contact, through the bag, permitting digital access to the bottom of a container or bottle is useful and sometimes necessary, for example, in securely tightening a cap onto a. bottle to preserve out-of-bag sterility. For this reason, a tray which has a through hole for bottle assembly and rigid containment about the mid-portion of the bottle provides significant advantages. For this purpose, it is an object to provide a tray, having holes where through a bottle is displaced and still firmly held thereat, as disclosed and claimed herein.

Novel methods and kits for sterilizing, filling and capping containers within a closed sterile environment must be varied by the specific requirements for doing such for each medicine being prepared using a kit made according to the instant inventions. As an example, fortified antibiotic eye drops generally require only a limited number of eye drop bottles, usually two to four, but, because effective eye drop bottles are usually not sufficiently clear to permit a visual inspection of a compounded drug in all cases, a clear, preferably glass, spare additional bottle may he provided for quality assurance purposes. Thus, by example, it is another object to provide such a kit with a plurality of fillable and cap-able containers, including eye drop bottles and a quality assurance bottle.

Medical preparations associated with administering Avastin is an excellent example of need for a special convenience kit. Avastin is delivered by injection into the eye using a medical syringe and needle rather than by drops. Due to the physiologic attributes of the site of an Avastin injection, it is required to use syringes which are silicone-free with both syringes and medicine sterilized to a predetermined SAL. Such syringes should be filled and stored under strict conditions before use. Also, there is a range of kit requirements associated with the number of steps required for syringe filling which should be considered and reduced where possible. It is therefore another object to provide a sterile kit in which one or more syringes are filled with a sterile product delivered in a sterile environment. It is another object to provide a kit made according to the present invention by which multiple syringes are filled with a single dispensing stop.

Preparations of medicines involving oncology or other hazardous drugs require special care for safety in handling as well as need for sterility. As an example, mitomycin preparation, for pad soaking, may require reconstitution of a lyophilized form of the drug along with providing the drug sterile in a closed and exteriorly sterile container such that it can be manipulated on a sterile field; it is an object to provide such.

Also, there is a matter of hazardous drug fume release when a vial containing a hazardous drug vial is spiked for fluid withdrawal. For this purpose, it is an object to provide a closed dual bag system kit whereby an oncology drug vial is disposed within a first bag, septum pierced and drug withdrawn therefrom, and delivered to a second bag for container filling through a closed pathway, whereby all hazardous drug remains enclosed until packaged for safety for later distribution and use. It is therefore an object to provide a sterile kit comprising two basic functions of enclosing a hazardous drug vial when piercing the vial septum and delivering the drug to an enclosed receiving vessel which is capped and provided closed before delivery of the drug to a place of use.

In the case of medicine delivered via syringe and needle, there are needs for priming gas from the syringe, while maintaining needle coverage until bared for immediate use and for restricting spread of syringe contents when priming. It is, therefore, an object to provide a priming cap for a medical needle and use thereof as disclosed herein.

Because kits made according to the instant invention are designed and made for specific compounding purposes, different sterilizing methods can be employed for addressing sterilization in each kit within the scope of the instant invention. As an example, light sterilization can be used within the scope of the instant invention, wherein fluid flowing into a hag is sterilized by light of a sterilizing frequency upon being displaced into the pathway into the bag. When conditions allow, heat may also be used within the scope of the inventions. However, currently, the most convenient and effective means of fluid sterilization is a medical grade (i.e. 0.2 micron) filter as disclosed in all examples provided hereafter. In summary, for all examples, disclosed herein, it is an object to provide in-flow sterilization into a sterile filling chamber via a sterilizing filter assembly.

For reasons mentioned supra and for providing devices and convenience kits which can be used to prepare product which meets USP Compounding Standards, the following objects of invention are hereafter provided.

It is a major object to provide a plurality of kits wherein each kit, selectable from other kits, has an associated sterility producing design which is proved effective for formulating a pre-specified medicine and made according to the instant invention. Therefore, each such sterile kit is characterized by at least one totally closed sterile chamber, which is pre-sterilized to a predetermined SAL; each sterile kit comprising fluid access only through a medical grade sterilizing filter such that all fluid dispensed into the sterile chamber is sterilized to a predetermined SAL. Thus, medicine formulated therein is sterilized and fully enclosed in vessels provided as part of a sterile kit which are capped and closed until provided for use. Each convenience kit (and associated sterile kit) is understood to be used just once.

It is a primary object to provide a convenience kit which can safely and effectively provide a method for producing and providing a plurality of syringes filled with medicine sterilized to a desired SAL, closed until used, for example, for dispensing Avastin.

It is another primary object to provide a convenience kit which is effective in filling and capping eye drop dispensing containers with an inherently sterilized preparation of fortified topical antibiotics within closed and sterile conditions.

It is still another primary object to provide a sterile kit for reconstituting a lyophilized drug disposed in a medical vial and displacing the reconstituted drug into a container disposed in a sterile environment, which is thereafter closed, and which meets sterile conditions for being sterile, once filled and closed. As such, drugs may be classed as being hazardous, it is also an object that an associated sterile kit be closed for drug access from a vial and further closed for delivery and filling of drug into use containers. An example of such a drug being mitomycin.

It is an important object to provide a bottle holding tray with a pattern of through holes for containing open and cap-able eye drop bottles within closed, sealed and sterilized environs, the holes being sized and shaped to resist bottle rotation as caps are affixed to the bottles but sized and shaped to provide digital access to the bottom of each bottle for better assurance of securely affixing cap to bottle.

It is a very important object to provide a needle cap for a syringe needle which provides a closed chamber wherein fluid is dispensed and held when an attached syringe is primed, further, it is preferred that the needle cap should provide visual contact of both needle tip and dispensed matter for feedback control while priming.

Thus, it is a global object to disclose, provide and claim a variety of convenience kits, each of which is used but once to formulate a specific medical preparation, sterilized to a predetermined SAL and made to be used for accomplishing a preparation having assured sterility and purity, examples of such being preparations of autologous blood serum eye drops, avastin, mitomycin, hazardous drugs and antibiotic fortified eye drops and provided in closed and sealed containers which, for example, can be eye drop bottles, medical syringes (sized and selected for each specific preparation), IV bag spikes and cap-able medical cups.

These, and other objects and features of the present inventions, will be apparent from the detailed description taken with reference to accompanying drawings.

### BRIEF DESCRIPTION OF THE DRAWINGS

FIG. 1 is a pre-assembly perspective of parts of a tray assembly for an eye drop processing kit.

FIG. 2 is an assembled perspective of the tray assembly seen in FIG. 1 rotated to show access to bottoms of bottles.

FIG. 3 is a perspective of a four-bottle tray assembly.

FIG. 4 is an exploded pre-assembly perspective of a bottle-plate assembly.

FIG. 5 is a perspective of parts in FIGS. 3 and 4 assembled.

FIG. 6 is a perspective of the assembly in FIG. 5 rotated to permit viewing of exposed bottle bottoms.

FIG. 7 is a perspective of the assembly seen in FIG. 6 disposed and sealed within a plastic bag according to the instant invention.

FIG. 8 is an exploded pre-assembly perspective of a bottle tray assembly with a bottle dedicated for quality assurance.

FIG, 8A is an exploded pre-assembly perspective of a bottle plate assembly.

FIG. 9 is a perspective of parts in FIGS. 8 and 8A assembled.

FIG. 10 is a top elevation of an unfilled syringe and a priming needle cap disposed within a sealed plastic bag having a fluid interface with surrounding environment through a filter assembly, according to the instant invention, some items being seen in cross section.

FIG. 11 is a top elevation similar to that of FIG. 10, but with the syringe filled.

FIG. 11A is a top elevation similar to that of FIG. 11, but with the syringe displaced from the filter assembly and affixed to the priming needle cap.

FIG. 12 is a top elevation schematic of a one-at-a-time syringe filling convenience kit wherein an array of frame-held syringes is seen with a first syringe freed from a medical needle and cap and displaced to connect with a filter assembly.

FIG. 13 is a top elevation schematic of the one-at-a-time syringe filling convenience kit seen in FIG. 12, but with the first freed syringe removed from the filter assembly and displaced to an attachment with an associated needle and needle cap.

FIG. 14 is a top elevation schematic of the one-at-a-time syringe filling convenience kit seen in FIG. 13 with a second syringe displaced and affixed to the syringe assembly.

FIG. 15 is a top elevation schematic of a convenience kit made according to the instant invention, a kit assembly is mostly enclosed within a clam-shell tray and is seen to comprise a plurality of empty syringes affixed to a manifold with a sterilizing filter also affixed to the manifold at an inferior site in the figure and a pair of fluid flow controllers superiorly affixed to the manifold.

FIG. 16 is a top elevation schematic of the convenience kit seen in FIG. 15A with a cover of the tray removed for clarity of presentation.

FIG. 16A is similar to FIG. 16 except the kit assembly is seen removed from the clam-shell tray.

FIG. 16B is similar to FIG. 16A except that a component has been interposed between each syringe and the manifold.

FIG. 17 is a top elevation schematic of the convenience kit seen in FIG. 16 with a filled syringe containing liquid to be displaced into kit assembly syringes via the manifold affixed to the sterilizing filter.

FIG. 18 is a top elevation schematic of the convenience kit seen in FIG. 17 wherefrom a portion of liquid disposed in the filled syringe is seen to be dispensed into the manifold.

FIG. 19 is a top elevation schematic of the convenience kit seen in FIG. 18 wherein contents of the syringe affixed to the sterilizing filter have been displaced into the manifold and therefrom to fill the plurality of syringes.

FIG. 19A is a top elevation schematic of the convenience kit seen in FIG. 19 wherein the plunger rod of the syringe affixed to the sterilizing filter is withdrawn slightly to draw liquid from the manifold.

FIG. 20 is a top elevation schematic of the convenience kit seen in FIG. 19A with the filling syringe removed from the kit assembly.

FIG. 21 is a top elevation schematic of the convenience kit seen in FIG. 20 with a top cover seen affixed to the bottom of the tray and displaced to provide access to filled syringes affixed to the manifold.

FIG. 22 is a perspective of a syringe used for communicating fluid through the vial spike assembly.

FIG. 23 is a top elevation schematic of an Avastin containing vial disposed within a plastic bag fitted with a vial spike assembly for spiking the vial and communicating fluid between the vial and the assembly.

FIG. 24 is a perspective of a hag with a filter assembly made according to the instant invention with a cup with a tethered cap affixed to a stabilizing tray disposed therein.

FIG. 24A is perspective of a bag with a filter similar to FIG. 24, but with the cup cap closed.

FIG. 25 is a perspective of the cup and tray seen in FIG. 24 disposed outside of the bag.

FIG. 25A is an enlarged perspective of the cup seen in FIG. 25 wherein a screen is seen supporting a pad away from the internal bottom of the vial.

FIG. 26 is similar to FIG. 25, but with the tethered cap disposed to close and seal the cup.

FIG. 27 is similar to FIG. 26, but with tray and cup turned upside down.

FIG. 28 is an open cup similar to the open cup seen in FIG. 25A, but with the pad seen disposed upon the tethered cap.

FIG. 29 is a perspective of a hazardous drug assembly made according to the present invention, the assembly comprising a zip lock vial holding bag which is fitted with a vial spike and fluid communication assembly affixed to a stopcock which is affixed to a syringe and to a filter assembly through which liquid is dispensed into a container within a closed bag apparatus made according to the instant invention.

FIG. 30 is a side elevation perspective of a medical vial which may contain a hazardous drug.

FIG. 31 is a side elevation of a schematic for kit made according to the instant invention similar to the perspective seen in FIG. 29 except that the vial, seen in FIG. 30, is disposed and spiked within the bag which has preferably been zip-locked prior to spiking of the vial.

FIG. 32 is similar to FIG. 31, but with the syringe being filled with liquid drawn from the vial.

FIG. 33 is similar to FIG. 32, but with the stopcock path controller displaced for liquid flow from the syringe to the filter assembly and thereby communicating liquid to the second syringe.

FIG. 34 is similar to FIG. 33, but with the second syringe disconnected from the filter assembly and with a priming needle cap, disposed within the associated bag affixed to the syringe.

FIG. 35 is a cross section of the needle cap seen in FIG. 22.

FIG. 36 is a cross section of the priming needle cap as seen in FIG. 23 wherein a free plunger disposed about a needle is displaced by priming fluid from an attached source.

FIG. 37 is a side elevation of a priming needle cap with internal parts indicated by dashed lines.

### DETAILED DESCRIPTION OF THE ILLUSTRATED EMBODIMENTS

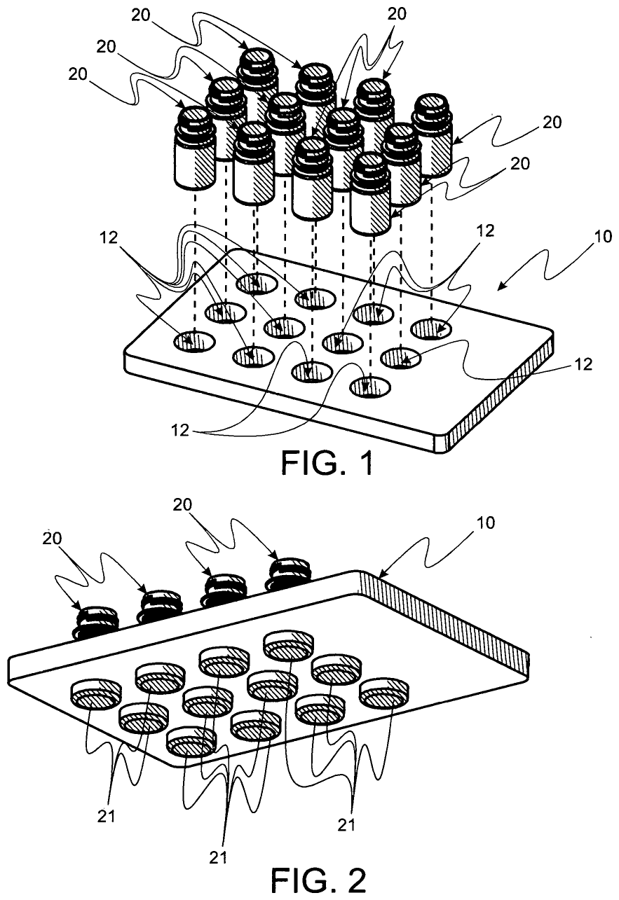

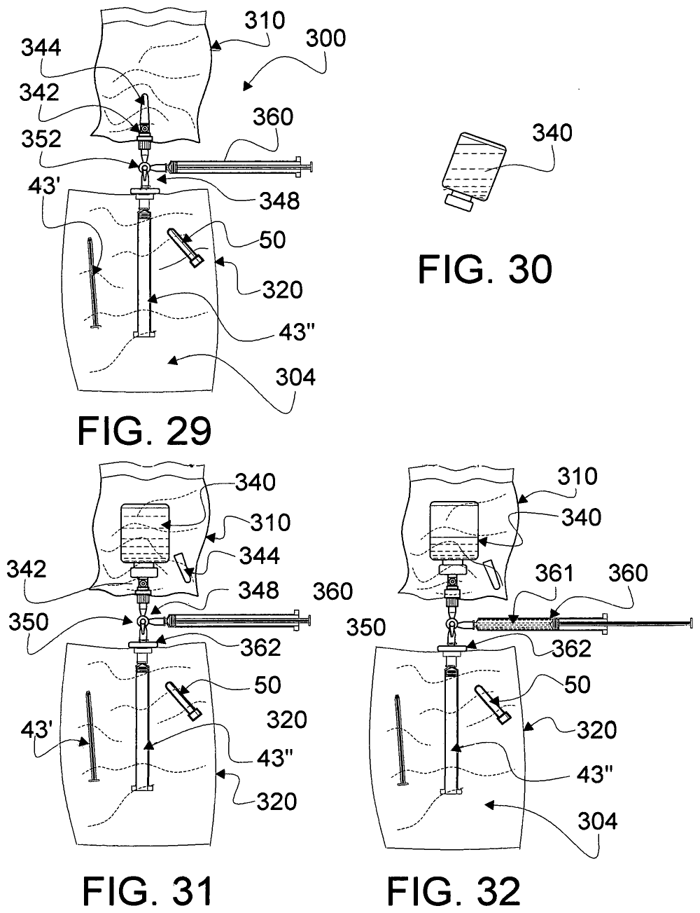

In this description, the term proximal is used to indicate a segment of a device normally closest to an object of the sentence describing its position. The term distal refers a segment oppositely disposed. Reference is now made to embodiments illustrated in FIGS. 1-40 wherein like numerals are used to designate like parts throughout. For parts which are similar but not the same as parts originally specified with a given number, a prime of the original numbers is used.

Kits disclosed herein are considered novel by specific application for use in preparing a particular medicine or medicine selected from a group of medicines. Reference is now made to FIGS. 1-9 wherein trays which employ a pattern of through holes are seen with a plurality of applications.

### Application: Autologous Blood Serum Eye Drops

As seen in FIG. 1, a tray 10 has a pattern of twelve through holes, each numbered 12. Tray 10 is preferably made from a crosslinked, closed cell foam which is sufficiently elastic to permit a bottle 20 to he displaced into a hole 12 which is smaller in diameter than the exterior diameter of a bottle 20 such that each bottle 20 is held fast and resists rotation. It is recommended that such trays be made from cross linked, closed cell foam which deters problems associated with tray generated particulates. Note that each through hole 12 permits access to a bottom 21 of a corresponding bottle 20 for facile removal from tray 10 as seen in FIG. 2.

### Application: Fortified Antibiotic Eye Drops

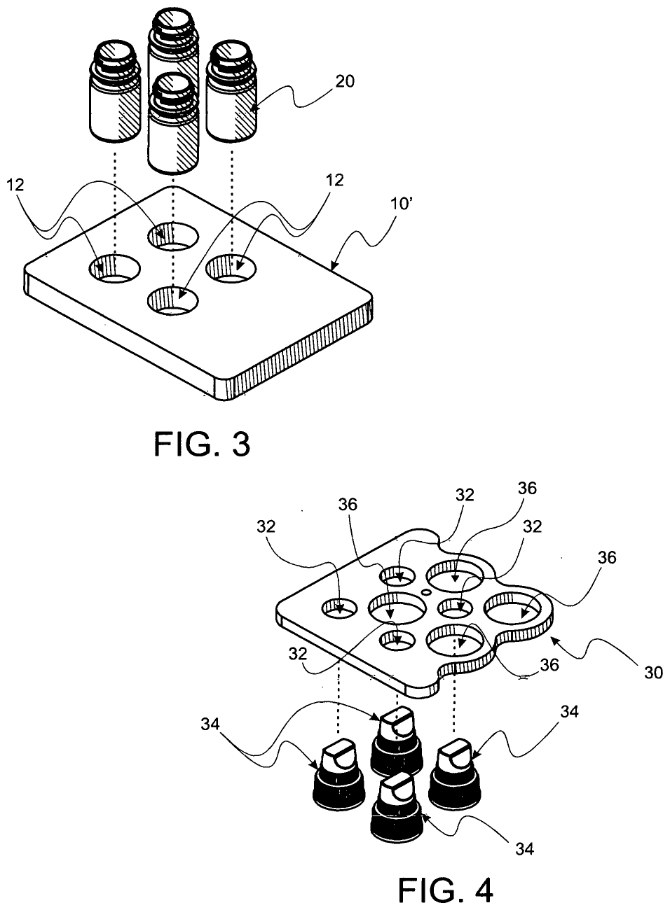

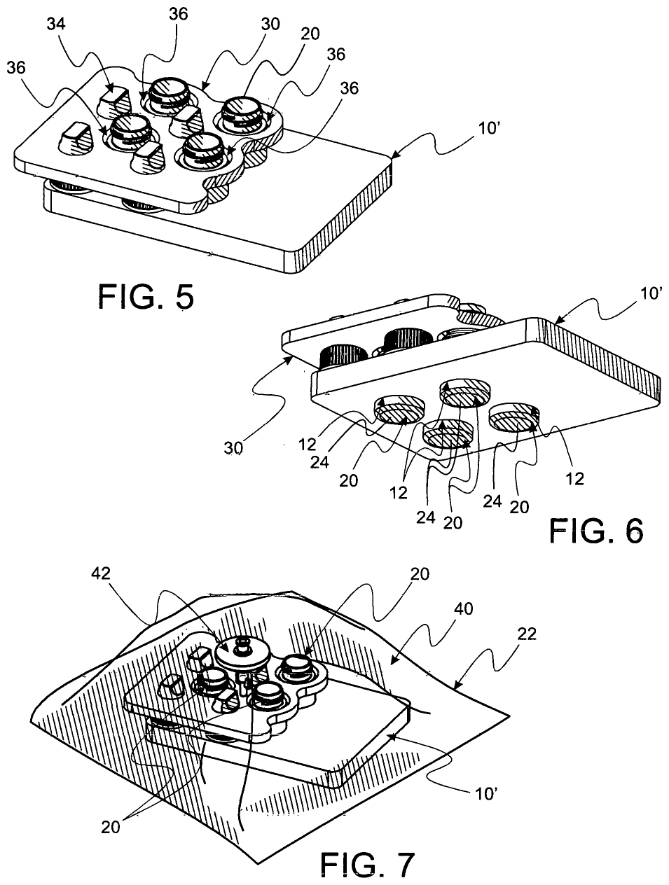

A more detailed example of a four bottle 20 and tray 10′ design is seen in FIGS. 3-7 for an exemplary sterile kit 22 (see FIG. 7). Similar to construction of plate 70 in U.S. patent application Ser. No. 16/873,780, a plate 30 (seen in FTG. 4) has two patterns of holes. A first pattern has holes, each numbered 32, sized and shaped to hold bottle caps, each numbered 34, for unitary cap displacement when displaced to cap bottles 20. A second pattern of holes, generally numbered 36, are disposed, sized and shaped to fit about bottles 20, as seen in FIG. 5. As seen in FIG. 6, where filled tray 10′ and plate 30 are rotated to make bottoms 24 of bottles 20 visible, through holes 12 provide digital access to bottle 20 bottoms 24 for securing caps 34 to bottles 20 as well as facilely displacing bottles 20 from holes 12.

The completed sterile kit 22, seen in FIG. 7, comprises:
1. A bag 40 containing a tray 10′ with bottles 20 affixed therein and plate 30, the bag being sufficiently pliant and rugged to permit bottle 20 filling and capping via only outside bag contact without contamination from outside bag 40;
2. Bag 40 being closed, sealed and sterilized along with all components resident therein;
3. A sterilizing filter assembly 42 which provides the only fluid pathway into the bag, the sterilizing filter assembly 42 being constructed in a manner similar to filter assembly 40 of U.S. patent application Ser. No. 16/873,780 to, thereby, provide for sterilized product filling of bottles 20 and for capping and sealing bottles 20 with caps before removal from bag 40. All sterilizing filter assemblies disclosed herein have similar construction.

As such, sterile kit 22 is specifically designed for use in filling and capping four eye drop bottles 20′ with fortified antibiotic eye drops. Each eye drop bottle 20′ for this application is recommended to have a volume of 10 mL. Various antibiotics can be effectively used, one at a time, in the general fortified antibiotic eye drop medication; examples of which are voriconazole, gentamicin, amikacin, cefuroxime, vancomycin and cyclosporine.

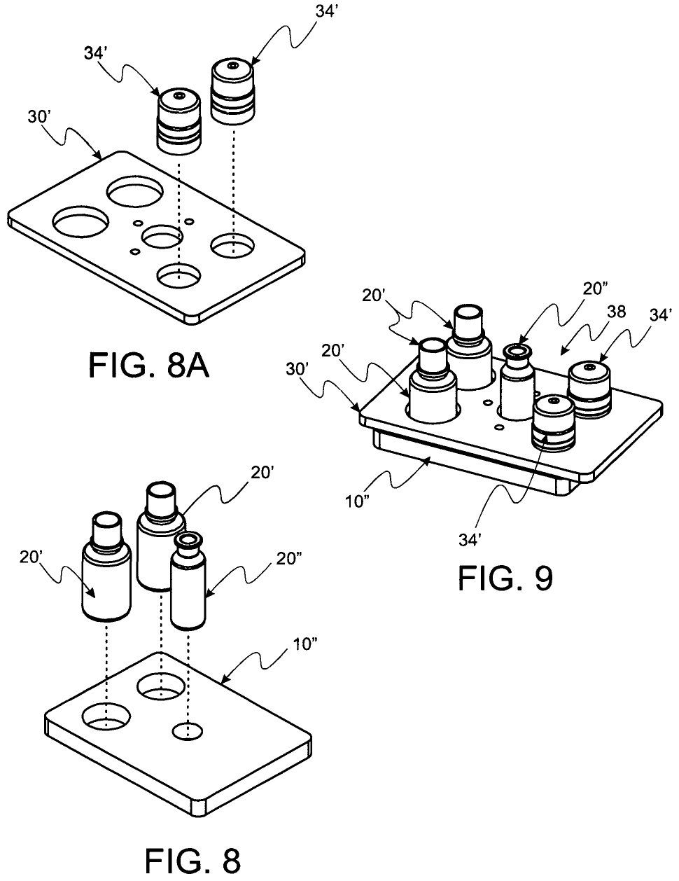

As the eye drop medication examples provided supra are simply examples and use can be much broader, it is understandable that a means for assuring quality of the resulting tears solution be quality assured. For this purpose, a clear material bottle 20″ provides opportunity for quality assurance testing, apart from bottles 20′ as seen in FIGS. 8, 8A and 9.

Referring to FIGS. 8, 8A and 9, a group of plates, trays, bottles and caps is seen to comprise:

1. Two eye drop bottles 20′ seen in FIG. 8.

2. A single clear, preferably clear glass bottle 20″ also seen in FIG. 8. Bottle 20″ is preferably cap-able to assure maintenance of purity. Such bottles are available commercially.

3. A plate 30′ which is similar in construction to plate 30, but patterned for two types of eye drop bottles, i.e. bottle 20′, bottle 20″ and associated caps 34′ seen in FIG. 8A.

4. A tray 10″ which is similar to tray 10′ but having a pattern for bottles 20′ and bottle 20″ delineated in item 3, above, is seen in FIG. 8.

Focus is now directed upon bottle 20″. Generally, eye drop bottles are not sufficiently clear for use in making visual quality assurance tests. As this kit may be used for a wide variety of antibiotic solutions, quality assurance of resulting tears solution and, therefore, clear bottle 20″, is worthy of note. An assembly 38 of of bottles 20′, caps 34′, bottle 20″, plate 30′ and tray 10″ is seen in FIG. 9.

### Application: Sterile Single Syringe Filling and Capping

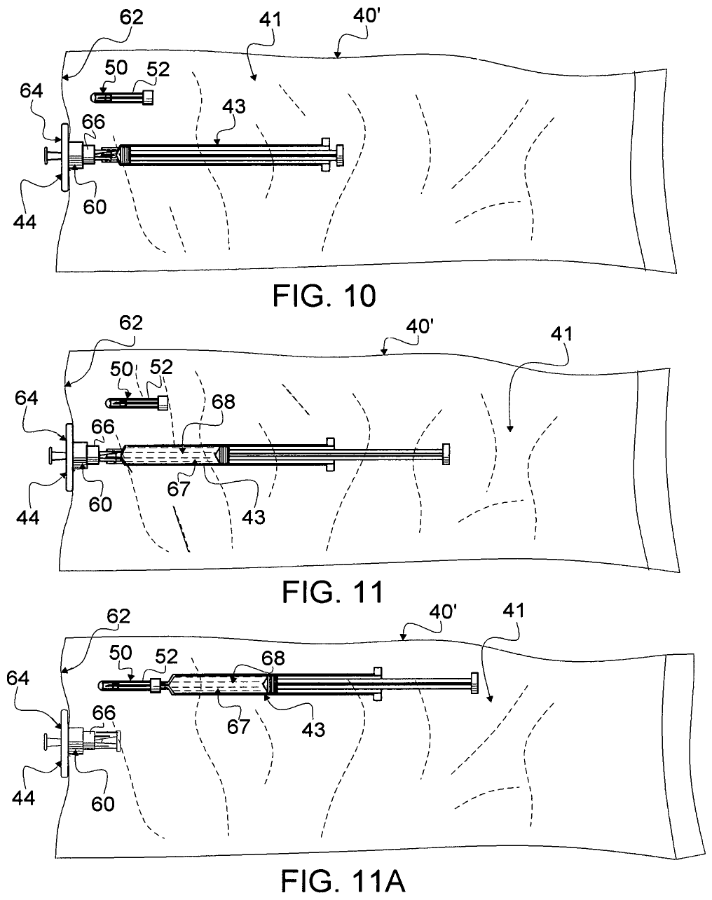

Reference is now made to FIGS. 10, 11 and 11A wherein, sterilized and enclosed within a closed and sterilized bag 40′ is a single syringe 43 securely, but releasably, affixed to a filter assembly 44 which provides the only fluid pathway into bag 40′ the interior of which provides an enclosed sterile chamber 41. As well, an unattached priming needle cap 50 which covers and shields a medical needle 52 is disposed as a free part disposed within bag 40′. A more detailed disclosure of cap 50 is provided hereafter.

Filter assembly 44 is similar in construction and function to other filter assemblies disclosed herein and in related art (e.g. filter assembly 42 (see FIG. 7)), however, in this case, filter assembly 44 is affixed to a closed end of bag 40′. A grommet 60 is tightly affixed against inside surface 62 of bag 40′ to close and seal about a hole in bag 40′ through which a female luer fitting of a sterilizing medical filter 64 is displaced to permit sterilized fluid sterilized and displaced through filter 64 to be delivered to syringe 43. The hole in bag 40′ and the female luer fitting are not shown in detail in FIGS. 10, 11 and 11A, but one who is skilled in medical art would well understand construction as disclosed in the related art referenced supra and the fitting being a commercially available item. Thus, a female fitting of filter 64 is securely affixed to the female luer fitting (not shown) of filter 64 to provide a connecting force against grommet 60 to close and seal the hole in bag 40′. Syringe 43 is securely, but releasably, affixed to filter assembly 44 via fitting 66.

As seen in FIG. 10, syringe 43 is affixed to female fitting 66 and provided empty within bag 40′. Once so affixed, hag 40′ is sealed and bag 40′ and all items disposed therein, including a priming cap 50, are sterilized. It should be noted that bag 40′ should be made of pliant plastic material which is sufficiently supple to permit syringe 43 to be rotated to become free of fitting 66 by only exterior contact with bag 40′. Such material should also be sufficiently hardy to permit such rotation without tearing the material.

As seen in FIG. 11, barrel 67 of syringe 43 is filled with a liquid medicine 68, sterilized when, as provided and delivered through filter assembly 44. However, once filled with a predetermined dose of liquid medicine 68, it is often preferable to cap syringe 43 before breaching hag 40′. Also, it is common to prime such a syringe before use. As seen in FIG. 11A, cap 50 provides capacity for priming without spilling syringe 43 contents into bag 40′ or other areas when syringe 43 is displaced from bag 40′. Further, disclosure concerning cap 50 is provided hereafter (see FIGS. 39-40A).

### Application: Multiple Syringe Filling and Capping

Reference is now made to FIGS. 12-14 wherein a multiple syringe kit 70 provided for a wide variety of uses, made according to the instant invention is seen. A plurality of medical syringes, generally numbered 72, are seen disposed and sterilized within a bag 74, which encloses a sterile chamber 75, as noted in FIG. 12, and therein affixed to priming caps 50 held firmly by a rack 76 with a single exception. This example is provided to show attachment of a priming cap, in those cases where each syringe is simply capped, rack 76 would provide a plurality of caps rather than priming needle caps.

The exception, a single syringe, also numbered 72′, is affixed to a filter assembly 78 made and affixed to bag 74 by a grommet 78 in the same manner syringe 43 is affixed to filter assembly (see FIG. 10). Note that bag 74 should have the same material character and quality as bag 40′, also seen in FIG. 10 and he formed with sufficient space for all activity disclosed herenin within sterile chamber 75. Other caps may be used within the scope of the instant invention but being able to prime syringes without spilling liquid therefrom is considered preferable. Also, plunger rods are not shown, as such can be affixed to displace each plunger after a syringe, generally numbered 72, is removed from bag 74 and bag 74 would have to be enlarged if plunger rods were initially so affixed. It should be noted that plunger rods can be affixed each associated plunger, generally numbered 82, and used without affecting the sterility state of each syringe 72 content. Such plunger rods and plungers 82 with accepting interfaces are well known in syringe art.

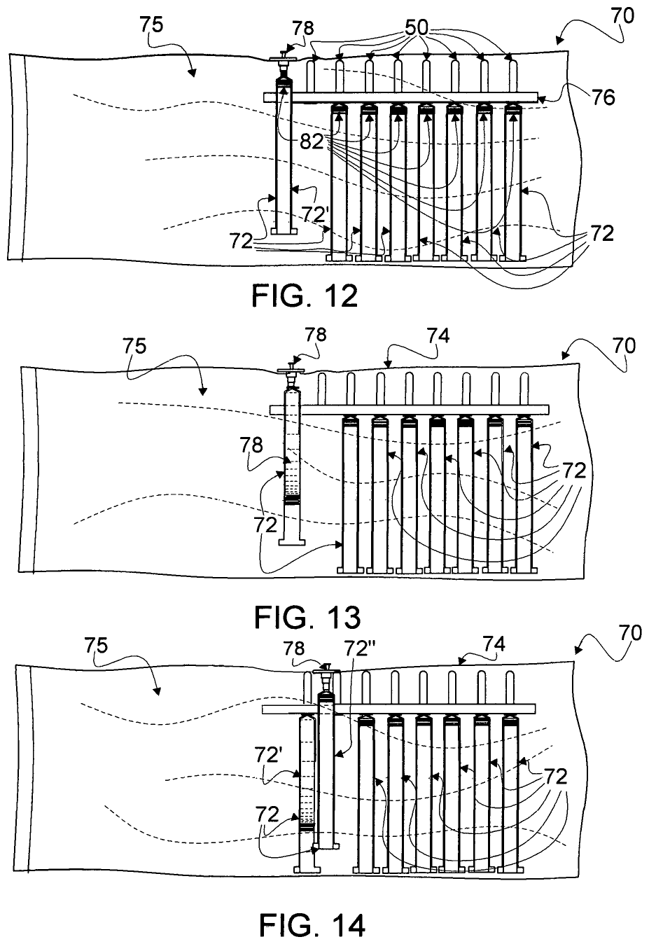

By example, kit 70 is seen to comprise eight syringes 82. Effectively, any number of syringes which will fit into bag 74 can be made part of kit 70. In FIG. 12, the single syringe 72, also numbered 72′, is removed from a cap 50 and affixed to filter assembly 44′ which is constructed similarly to filter assembly 44. Note, that all syringe handling is effected by syringe contact via hag 74 to maintain product sterility. So affixed, syringe 72′ is filled with a predetermined volume of liquid 78 as seen in FIG. 13.

Thereafter, filled syringe 72′ is removed and reaffixed to an available cap 50. A second syringe numbered 72″ is then removed from a cap 50 and digitally affixed to filter assembly 44′ and thereat filled with a predetermined volume of liquid. This process is continued until all syringes have been filled with a desired volume of sterilized liquid 78 in the same manner syringe 72′ was filled. Once such has been accomplished, bag 74 (i.e. sterile chamber 75) can be breached for access to capped syringes 72 filled with a sterilized product.

### Application: Sterile Single Step, Multiple Syringe Filling

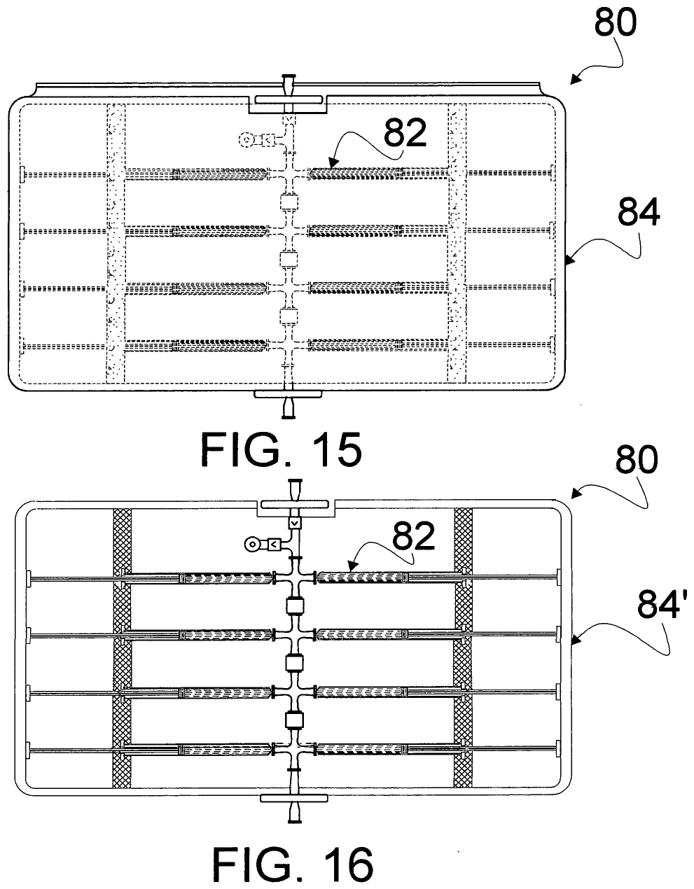

Method and convenience kit for filling multiple syringes in a single step is provided, by example, using a convenience kit 80 (see FIG. 15) made according to the instant invention as se in FIGS. 15-21A. As seen in FIG. 15, a core portion 82 (the “sterile kit assembly”) is disposed within a porting tray 84, contents of which are seen as dashed lines. Portion 82, being a closed filtering and dispensing portion of kit 80, is seen separate from tray 84 in FIG. 21A. Portion 82 comprises a medially disposed manifold 90 made up of one cross connection fitting 92 for each pair of syringes (each syringe being numbered 94) affixed thereto. In this example, fittings (commonly numbered 92) of manifold 90 are interconnected by male/male luer fittings (commonly numbered 96). One connecting fitting 97 of an inferiorly disposed cross connection fitting 92 is interconnected with a filter 64′, functionally the same as filter 64, disclosed supra. In this case, the interior pathway of manifold 90 is the sterile chamber, not shown, but well understood by those who are skilled in medical device art.

On an end 98 of manifold 90, a female fitting 98′ (not shown) is affixed to a “T” connector 99 providing a first pathway to a one-way valve 100 which is permissive to flow out of manifold 90 to an air release/liquid retaining valve 102. Such valves are commercially available. One skilled in manifold design would understand character of a pathway through manifold 90 tracing a pattern from filter 64′ to each syringe 94 and to pathway end “T” connector 99, also available commercially. An ascending leg 104 of “T” connector 99 is affixed to another one-way valve 106 which is disposed to only permit fluid flow into manifold 90. Another filter 64″, similar in form and function to filter 64′ is affixed to one-way valve 106 to provide a filtered pathway for air entry into manifold 90 when pressure gradients within manifold 90 are appropriate.

Each syringe 94 has a barrel (commonly numbered 110) and a plunger (commonly numbered 112) securely affixed to a plunger rod (commonly numbered 114). Also, each barrel 110 has a bulbous open end (commonly numbered 116). As well, each plunger rod 114 has an end pad (commonly numbered 118). Bulbous end 116 and pad 118 are commonly used for digital displacement of plunger rod 114 relative to barrel 110. On fluid communication dispensing end 119 of filter 64′, is a male luer fitting 119 (not completely shown, but well understood by those skilled in medical syringe art). In core portion 82, syringe associated luer fittings are seen to be luer-slip (as opposed to “luer-lock”), as is commonly the case for small syringes, such as those used for Avastin. Due to the size and function of each syringe in an Avastin medical treatment it is often preferable that such fittings be luer slip. For Avastin, syringes use a generally small (i.e. about 1 mL and must be non-siliconized. Note that when core portion 82 is sterilized, the manifold pathway remains sterile due to the only inflow pathway openings being by interconnecting filters 64′ and 64″. As such, core portion is pre-sterilized and provided sterile with empty syringes 94 affixed at the time of sterilization, as seen in FIG. 17. It should be noted that in the case of core portion 82, a filling chamber is not a bag, but the pathway through manifold 90.

The filling process is as follows:

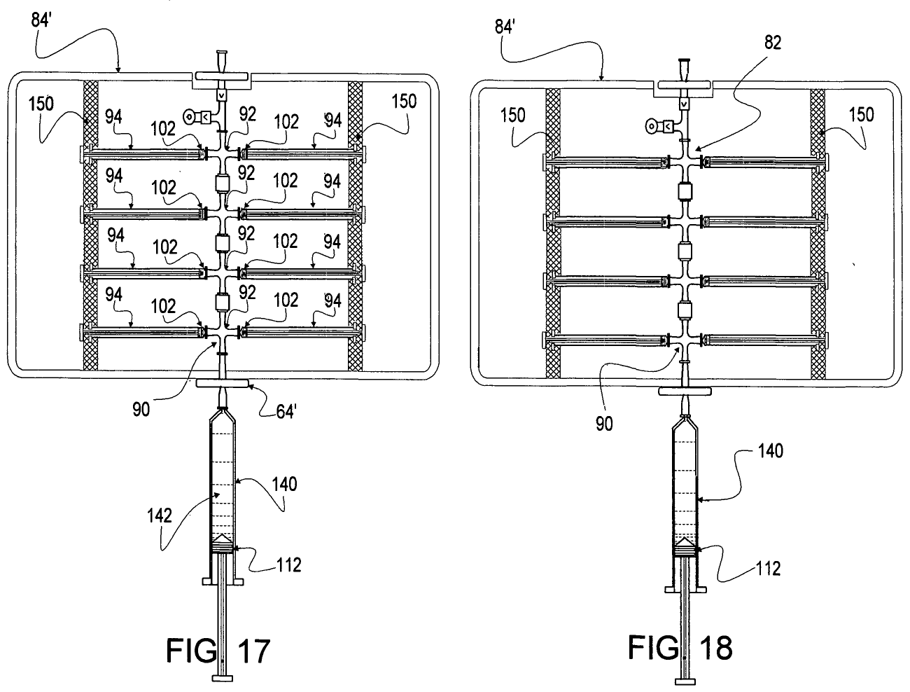

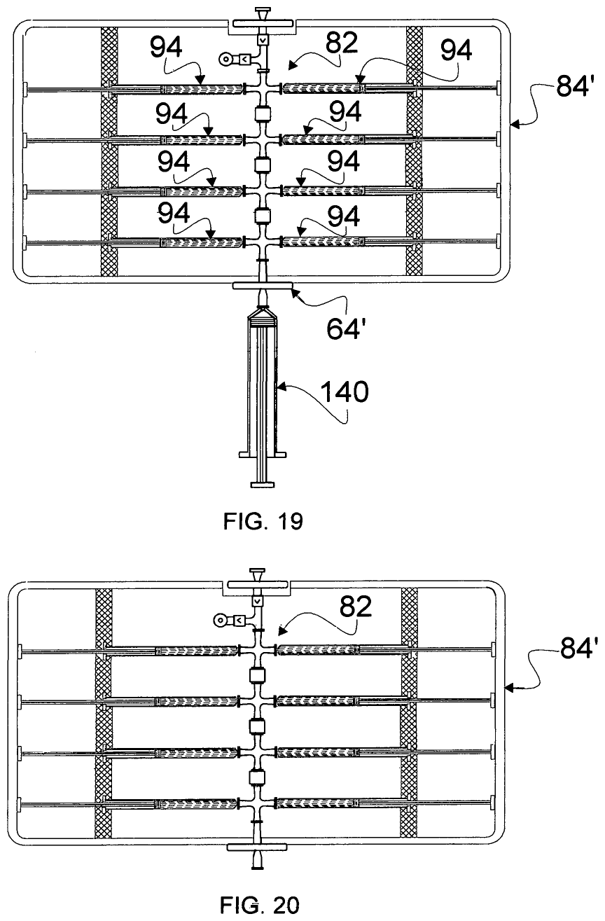

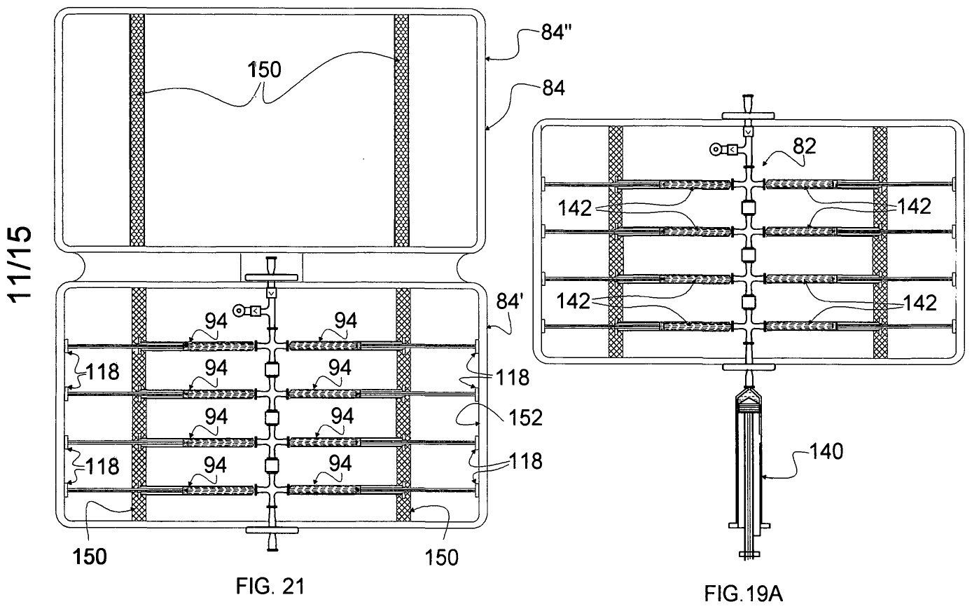

As seen in FIG. 17, a source syringe 140, filled with liquid medicine 142 to be displaced into syringes 94 is affixed to filter 64′. To minimize gas dispensed into syringes 94, the first liquid displaced from syringe 140 fills manifold with gas being driven outward through valve 102 (the lowest resistance pathway) until liquid reaches 102 which requires delivery of a very small amount of liquid as seen by limited displacement of plunger 112 in FIG. 18. Once air flow is complete and fluid flow is thereby prohibited by valve 192, syringe 94 filling begins. Continued displacement of liquid 142 from syringe 140 fills syringes 94, as seen in FIG. 19.

As seen in FIG. 21, tray 84 is preferably of “clam shell” design with having a bottom 84′ and a top 84″ (see FIG. 21). As seen in FIG. 17, syringes 94 are provided empty. When liquid is introduced into manifold 90 through filter 64′, stiction in one or more plungers 102 may result in a higher dispensing pressure than retention force, between manifold 90 and an associated syringe 92 than the connection to associated manifold 92 fitting can withstand, resulting in syringe 94 and fitting 92 separation. Such separation is not acceptable.

To obstruct such separation, restraints in the form of strips of firm, but impressionable material can be used if each syringe 94 is not fitted with a luer lock fitting. As an example, four strips (commonly numbered 150) of closed cell foam having thickness adequate to be indented by bulbous end 116 and thereby be restrained from being displaced when covered and compressively restrained by top 84″ and bottom 84′ of tray 84.

Further to impede overfilling of syringes 94, a raised inside edge 152 is sized and shaped to provide a stop for each plunger rod 118. Using strips 150 and inside edges 152, syringes can be filled without incident. Completion of a filling cycle, with syringe 140 emptied is seen in FIG. 19A.

In summary, kit 80 is provided as seen in FIG. 15 as a clam shell tray 84 with sore portion 82 disposed therein. A syringe 140 filled with medicine to be sterilized and displaced into syringes 94 is affixed to filter 64′ as seen in FIG. 17. The first liquid dispensed from syringe 140, as seen in FIG. 18 primes air from manifold 90, as depicted in FIG. 18. Syringes a filled by displacing medicine from 142 syringe 140 into syringes 94, as seen in FIG. 19. Liquid resident in manifold 90 is withdrawn therefrom by drawing liquid back into syringe 140 and replacing such liquid with air delivered through filter 64″ as seen in FIG. 19A, such will allow unitized core portion 82 to be frozen as a unit for storing and maintaining quality of medicine 142 until thawed and used. Once syringe 94 filling is complete syringe 149 can be removed as seen in FIG. 20. Cover 84″ seen in FIG. 21 is closed for storage if tray 84 is so used. However, core portion 82 can be displaced form tray 84 at any time after filling, being unitized as seen in FIG. 21A. Thereby access is provided to each syringe 94 as needed.

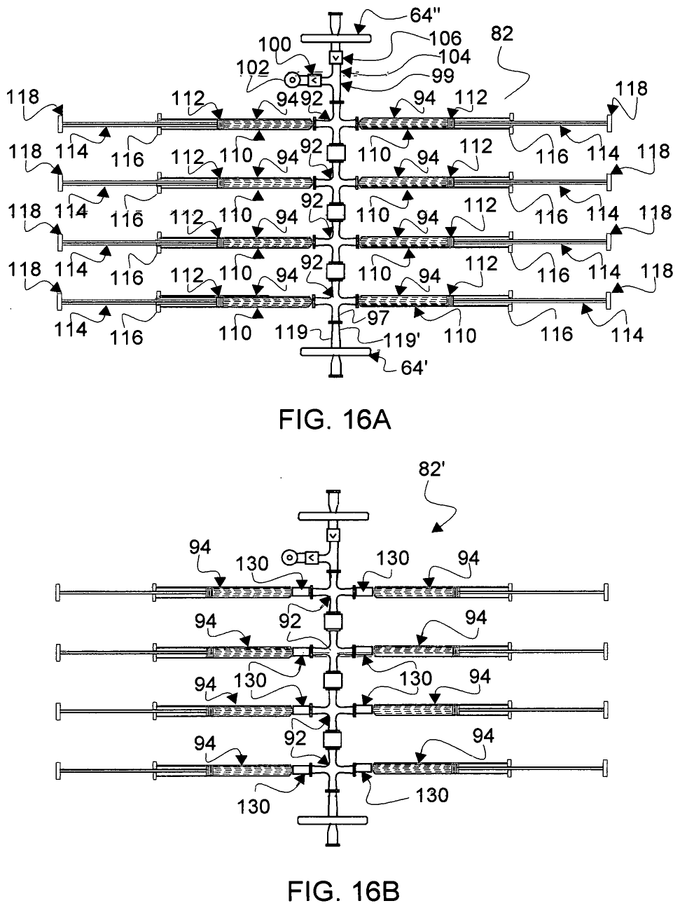

As displacing a syringe 94 from portion 82 opens a pathway from a potentially contaminated environment into the sterile chamber, even though likelihood of contamination of remaining syringes 94 being resultingly contaminated is slight, though possible. By adding contamination blocking devices, generally numbered 130, displaced between each syringe 94 and associate fitting 92 as seen in portion 82 in FIG. 16B, such likelihood of remaining syringes being contaminated is eliminated. Such blocking devices may be needleless connectors or sterilizing grade filters.

### Application: Mitomycin Sterilization and Pad Soaking

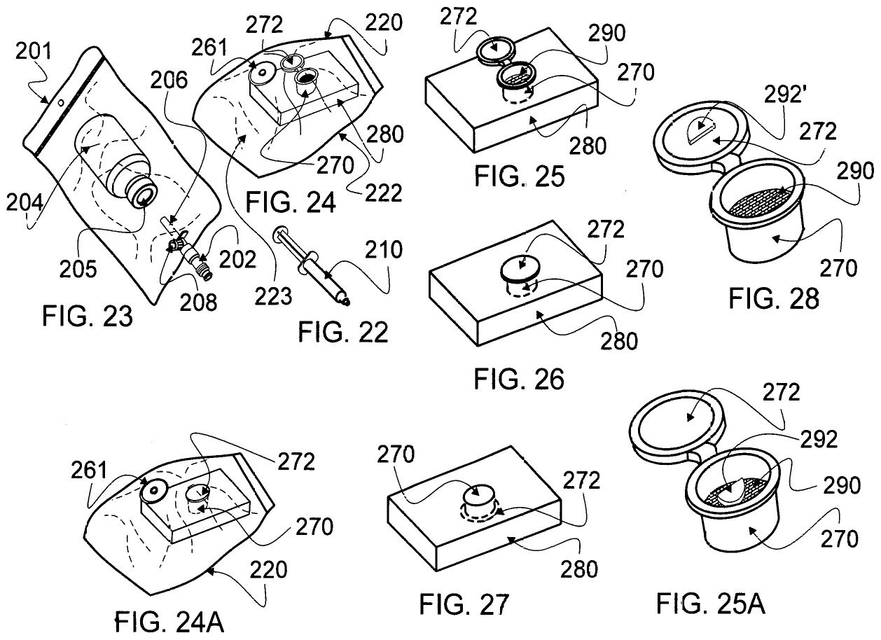

Reference is now made to a combination of kits seen in FIGS. 23 and 24 and a syringe seen in FIG. 22 which form a combination of kits and a syringe for being used for sterilizing mitomycin and soaking pads for applying sterilized mitomycin in surgical procedures according to the instant invention. U.S. Pat. No. 9,449,521, titled METHODS FOR MAKING AND USING A VIAL SHIELDING CONVENIENCE KIT discloses a plastic bag (which may be a Ziplock bag) for accessing a hazardous drug. While it is common practice to spike a mitomycin vial without protective cover, placing a cap-able cup in a sterilized plastic bag 201 with a liquid access assembly 202, as seen in FIG. 23, provides two elements of safety by pre-sterilizing bag 201 and assembly 202 to reduce likelihood of drug contamination and by zip sealing bag 201 after placing a vial 204, therein, after removing a septum cover and sterilizing the associated septum 205 thereafter, which assures maintenance of a sterile spiking interface.

Vial 204 is seen disposed within bag 201 in FIG. 23, whereby safety is increased by guarding against fumes released upon spiking and maintaining sterility of all spiking interfaces. Note that assembly 202 has a spike cap 206 which is displaced from covering a spike after vial 204 is disposed in bag 201. As it is known that vial 204 can be spiked with only digital access from outside bag 201 and that assembly 202 comprises a vent 208 for a spike, vial 204 can be spiked for both introduction of liquid into vial 204 and for drawing liquid from vial 204.

A syringe 210 seen in FIG. 22 provides for introducing a diluent into vial 204 when mitomycin is provided as a lyophilized solid and for withdrawing liquid mitomycin from vial 204. A second bagged kit 220 is seen in FIG. 24. Kit 220 comprises a closed bag 222 fitted with a filter assembly 261 whereby all fluid delivered into kit 220 is sterilized by a 0.20 micron filter. Any upstream contamination is thereby dispelled before dispensing the mitomycin into a delivery vessel disposed within a sterile chamber 223, disposed within bag 222.

In this example, mitomycin is dispensed through filter assembly 261 into a 3 ml cap-able cup 270. As a normal dose of mitomycin is approximately 1 ml, the 3 ml cap-able cup 270 is well suited for this application.

Cap-able cup 270 has a tethered cover 272 which seals contents therein when cap-able cup 270 is closed. Such cap-able cups are available as medical cap-able cups commercially. Further, cap-able cup 270 is firmly disposed into a closed cell tray 280, also disposed within sterile chamber 223, as seen in FIG. 24.

Tray 280 acts as a stabilizer for facile interfacing between filter assembly 261 and cap-able cup 270. As kit 220 is sterile along with contents therein, once cap-able cup 270 is provided with a dose of mitomycin, tethered cover 272 is displaced to cap and seal contents of cap-able cup 270 in place. With tray 280 also being sterilized, both cap-able cup 270 and tray 280 can be displaced from bag 222 onto a sterile surface without fear of contamination. Note that cap-able cup 270 is seen capped and ready for being accessed to a sterile surface in FIG. 24A.

As mitomycin is provided within cap-able cup 270 for soaking one or more pads for a medical procedure, contents and use of cap-able cup 270 follows. As seen in FIG. 25, cap-able cup 270 has a liquid permeable screen 290 disposed above the interior bottom (not seen) of cap-able cup 270. Screen 290 is preferably disposed sufficiently far above the interior bottom that the dose of mitomycin is fully disposed between screen 290 and vessel bottom.

To soak a pad, a pad 292 is placed upon screen 290 as seen in FIG. 25A and cover 272 is closed as seen in FIG. 26. Then tray 280 and cap-able cup 272 are turned upside down as seen in FIG. 27 to soak pad 292. After sufficient soak time has passed, tray 280 and cap-able cup 272 are turned again upright as seen in FIG. 26. When cover 272 is displaced to an open state, a soaked pad 292′ is likely found either on screen 290 of affixed to cover 272, as seen in FIG. 28. Of course, within the scope of the instant invention, more than one pad 292 can be soaked at a time.

### Application: Hazardous Drug Handling and Displacement

Reference is made to FIGS. 30-34 wherein an example of a hazardous drug handling and displacement kit 300 made according to the present invention is seen. An example kit, made according to the instant invention and provided for use in hazardous drug handling and transferring for use is seen if FIG. 30 where such a kit 300 is seen prepared for use. When preparing medicine using a hazardous drug, consideration must be given to both preparatory devices used in acquiring liquids from a drug source and to the purity and sterility of the final product. For this reason, two plastic bags, numbered 310 and 320 are provided as kit parts.

Both plastic bags are sufficiently pliant for manipulation of bag contents by contact with the bag exterior and made of sufficiently rugged material to endure such manipulation without being breached. Material of both bags is preferably sufficiently clear so contents can be viewed during manipulation.

Bag 310 is a source bag and provided for containment of spilled liquids and fumes resulting from access to a source container. A vial 340, seen in FIG. 34A, is provided as an example. Bag 310 is preferably a zip lock bag which is fully closed when zipped with only a single fluid pathway being provided into and out of bag 310 via a vial spike assembly 342 which is affixed through a hole in the bag (not shown), the hole being closed and sealed in a manner similar to closing a hole about a filter assembly as disclosed supra. Spike assembly 342 preferably has a conventional bag spike (not shown) which is covered and protected by a spike cover 344.

Interconnection assembly 348 in this example, employs a stopcock 350 and a medical syringe 360 assembled and may be affixed to be unitized with the bag spike assembly 342. However, if syringe 360 is employed to provide a diluent for contents of vial 340, syringe 360 may be affixed at the time of use of kit 300. It is important to note that, once affixed, syringe 360 should remain affixed and so held when kit 300 is disposed. Stopcock 350 is further unitized with a filter assembly 362, associated with bag 320.

Filter assembly 362 and bag 320 are similar in form and function to filter assembly 44 as seen in FIG. 10, except that a plunger rod 43′ is provided free from syringe 43″ within bag 320. It is common in the medical syringe art to provide a plunger rod free from an associated syringe when extension of the plunger rod upon syringe filling would make an excessively long apparatus. Therefore, plunge rod 43′ is provided separate from the rest of syringe 43″ for later introduction and use. As well, a free cap, which is preferably a cap 50, described and disclosed in detail hereafter, is enclosed within bag 320.

As a first step following institutional protocol, vial 340 is displaced into bag 310, bag 310 is zipped closed and vial 340 is spiked for liquid communication with stopcock 350 as seen in FIG. 31. Note that stopcock 350 is preset for communication between syringe 360 and vial 340. As seen in FIG. 32, liquid 361 is drawn from vial 340 into syringe 360 as a truly closed system operation.

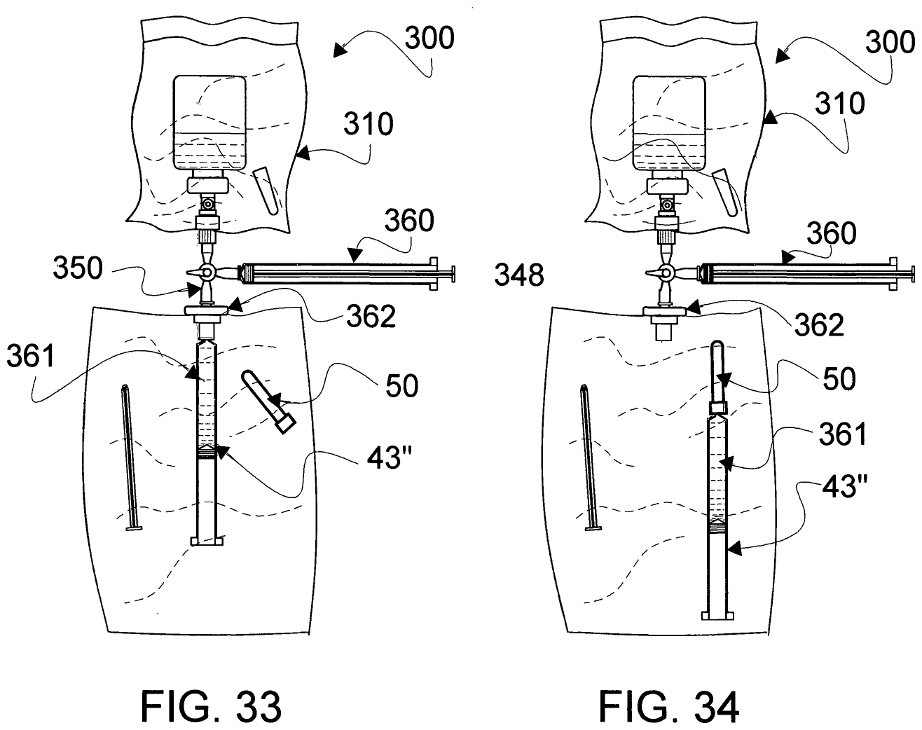

Stopcock 350 is then switched to direct flow between syringe 360 and syringe 43″ whereby liquid 361 is displaced into syringe 43″, as seen in FIG. 33. Once displacement is complete, syringe 43″ is displaced from filter assembly 362 through associated filter chamber 364 by only manipulations to the outside of bag 320. Syringe is then capped with priming cap 50 as seen in FIG. 34 and, with liquid 361 fully enclosed for sterility and safety, bag 320 may be breached with safety for access to filled syringe 43″.

### Application: A Syringe Needle Cap for Spilless Syringe Priming

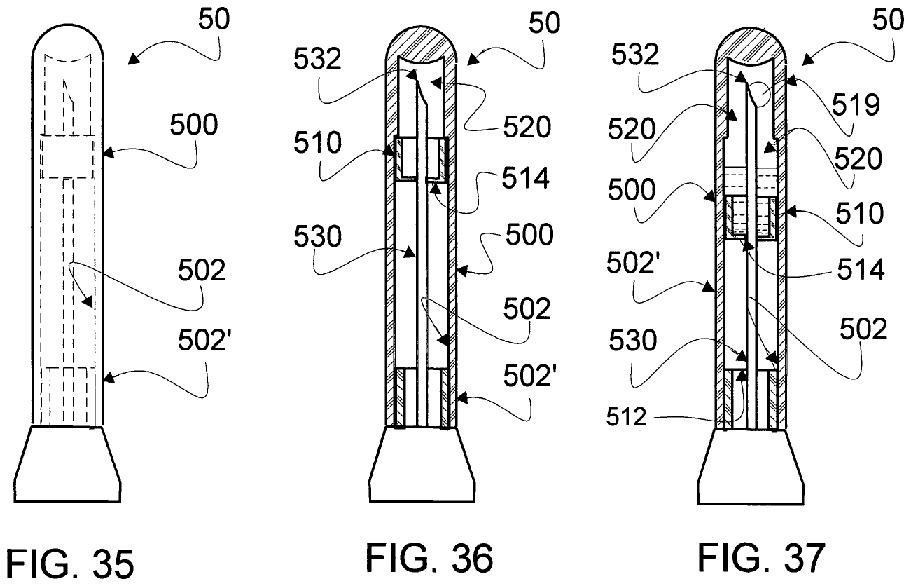

Reference is now made to FIGS. 35-37 wherein apparatus and function of a priming needle cap 50 is seen. As seen in FIG. 22, cap 50 comprises a substantially constant exterior and interior diameter barrel 500 which has a standard needle hup and luer fitting interface (not shown) disposed for affixing cap 50 to a convention luer fitting syringe. As seen in FIG. 35, cap 50 further comprises a cylindrical internal barrel surface 502 which is similar, but diminished in size, to the barrel 500 exterior surface 502′. Disposed within and residing against surface 502 in a manner similar to the way a syringe plunger is held within a syringe barrel is a needle pierceable plunger 510, best seen in FIG. 36. Plunger 510 is preferably made from material having the same piercing qualities as drug vial septa. Plunger 510 is sized and shaped to be displaced along surface 502 to a stop 512 molded with a reduced internal diameter at a predetermined place along surface 502 to provide an empty chamber 520 when disposed against stop 512. Further, plunger 510 has a septum 514 which is pierced by a needle (sharpened cannula) 530 as best seen in FIG. 36 with a needle point 532 disposed within chamber 520.

With plunger 510 disposed as seen in FIG. 36, a syringe requiring priming is affixed to cap 50 (not shown, but similar practice occurs commonly daily in medical facilities). Generally, syringe priming is meant to rid a syringe of air, hut the process usually results in a bolus of liquid being uncontrollably dispensed. If such liquid is hazardous in any way, such action may be unsanitary, if not dangerous.

As seen in FIG. 37 priming action displaces both dispensed gas and liquid 519 into chamber 520 where it is contained. Note that chamber 520 expands as fluid is displaced there into. For better control of priming action, cap 50 may be molded of clear plastic with a lens 550 disposed near a site of fluid ejection (i.e. needle point 532), as seen in FIG. 37, the focal point of the lens being needle point 532.

What is claimed and desired to be secured by Letters Patent is:

1. A method for providing an inventory of convenience kits made according to the instant invention, for sterilizing, displacing medical preparations into sterilized containers disposed in isolated communication with a sterile chamber and closing the containers for sterility retention, and for selecting at least one of the convenience kits from the inventory of the convenience kits, each convenience kit, within the inventory, being specifically destined to be used, but once, for preparation of a particular medicine selected from a group of medicines required to be provided as sterile preparations, said group of medicines comprising autologous blood serum tears, Avastin, hazardous drugs and fortified antibiotic eye drops, said method comprising the following steps:
a. providing said inventory of convenience kits, each of which comprises a closed sterile kit comprising a filter assembly, which provides the only fluid entry pathway into the closed sterile chamber within said sterile kit, for sterilizing the particular medicine formulation to a predetermined SAL, said sterile chamber, being disposed within said sterile kit and being a transfer pathway through which the medical formulations are displaced and delivered via said filter assembly into a predetermined number of pre-sterilized, close-able containers in communication with said sterile chamber, said containers having been selected from a group of containers comprising eye drop bottles, medical syringes and cap-able cups and being vessels into which the medical formulations are displaced, each container being pre-sterilized and pre-disposed, for being filled, closed and sealed with contact only being made to the exterior of the sterile kit, before the containers are dispatched from the sterile kit, thereby providing protection against contamination of the preparation when displaced into a potentially contaminating environment;
b. determining the particular medicine to be formulated;
c. selecting and acquiring a specific convenience kit, from the group of convenience kits, each such kit also comprising any items needed for formulating the particular medicine;
d. formulating and preparing the particular medicine;
e. dispensing the prepared particular medicine through said filter assembly into said chamber and displacing a predetermined volume of the particular medicine into each of the containers;
f. closing and sealing the containers while resident within the sterile kit; and
g. thereby providing a closed, packaged preparation of the particular medicine which is sterilized to a predetermined SAL for use in a potentially contaminating environment.

2. A method according to claim 1 wherein the determining of the particular medicine to he formulated step determines a specific method for formulating, sterilizing, filling and capping at least one eye drop bottle with fortified antibiotic eye drops using a kit made according to the instant invention, said method comprising the following steps:
a. selecting a convenience kit specifically made according to the instant invention for filling eye drop bottles with formulated fortified antibiotic eyedrop medicine, said convenience kit comprising a bag assembly which comprises said sterile chamber within a bag made of plastic material which is amply pliant for digital manipulation, but sufficiently rugged to effectively resist being perforated by such manipulation to assure there is no contact by any contaminant exterior to said bag assembly, said bag assembly being fitted with said sterilizing filter assembly affixed to the bag to therewith provide the only fluid pathway into said sterile chamber of said bag assembly and by which all fluid is sterilized when introduced, into said bag assembly, there through, said bag assembly further comprising paraphernalia comprising at least one eye drop bottle, into which liquid is dispensed to provide the sterilized formulated medicine, and a cap for each said at least one bottle for capping after bottle filling, before said at least one bottle is accessed via bag opening, to thereby preserve medicine sterility when the capped bottle is removed from the bag assembly;
b. acquiring and displacing each formulating ingredient required for formulating the particular fortified eye drop medicine into a dispensing, apportioning device;
c. affixing the dispensing, apportioning device to said filter assembly and, thereby, filling the bottles disposed, as prescribed, by displacing said particular medicine into said sterile chamber;
d. securely affixing a cap to each at least one bottle to provide a sealed fortified antibiotic eye drop product for delivery into a potentially contaminating environment with assurance of retained, predetermined, sterility; and
e. breaching the bag for access to the so capped bottles filled with so formulated medicine being provided at a pre-determined SAL.

3. A method according to claim 2 wherein an additional step of premixing, at least a portion of, the acquired ingredients of the medicine is performed before the bottle filling step such that a resulting mixture is resident in an apportioning device before being dispensed through said filter assembly.

4. A method according to claim 1 wherein said determining the particular medicine to be formulated and selecting and acquiring the specific convenience kit steps comprise selecting a convenience kit made according the present invention comprising a sterile kit for filling eye drop bottles, said sterile kit comprising a bottle holding tray as an item of said paraphernalia, said tray further comprising a pattern of through holes sized and shaped to be a container for eye drop bottles which securely preserves bottle upright status for filling and capping and further provides through bag digital access to the bottom of each bottle, for securely affixing caps to bottles to thereby assure a fluid tight seal.

5. A method according to claim 1 wherein said filter assembly providing step comprises providing a 0.2 micron filter sterilizing apparatus.

6. A convenience kit according to claim 1 wherein said paraphernalia providing step comprises providing at least one bottle dedicated for use in quality assurance testing of the formulated medicine.

7. A method according to claim 1 wherein said medicine determining step comprises determining Avastin to be the medicine and wherein said kit selecting and acquiring step comprises selecting and providing a kit specifically designed and qualified for compounding and sterilizing Avastin medicine and displacing the medicine into silicone free syringes.

8. A method according to claim 7 wherein said kit comprises a step for selecting and acquiring a pre-sterilized Avastin preparation kit whereby said kit selecting step comprises selecting an Avastin preparation kit comprising:
a. a filter assembly which provides the only pathway into said kit;
b. a manifold affixed to said filter assembly and comprising a closed connecting pathway and a syringe fitting for each syringe to be affixed thereto;
c. a silicone free syringe each with an associated plunger being disposed to provide an empty syringe barrel affixed to each said fitting; and
d. closing apparatus affixed at an end of said manifold distal from the filter assembly to assure maintenance of pathway sterility.

9. A method according to claim 8 comprising a further step of providing closing apparatus comprising providing an air elimination valve in line with a one-way valve which only permits gas to he delivered from said pathway when liquid is dispensed therein to assure gas initially resident within the pathway is purged from said manifold.

10. A method according to claim 9 comprising a further step of providing an apportioning device filled with Avastin medicine for dispensing and filling each said syringe affixed to said manifold.

11. A method according to claim 10 further comprising filling each said affixed syringe by but a single step of digitally actuating the apportioning device to dispense the Avastin medicine through the sterilizing filter and pathway into each syringe.

12. A method according to claim 9 comprising a further step of providing closing apparatus comprising an additional filter assembly affixed to a one-way valve which is permissive to fluid flow into said pathway.

13. A method according to claim 12 comprising an additional step following said syringe filling step comprises drawing liquid resident in said pathway such that the liquid is replaced by air flowing through the additional filter assembly and associated one-way valve to remove the liquid from said pathway to minimize effects of liquid expansion upon freezing the remaining Avastin medicine for long term storage.

14. A method according to claim 7 wherein said kit providing step comprises providing a tray wherein said kit is disposed while syringes are filled, said tray comprising a barrel restraint for each syringe to assure maintenance of syringe and manifold connection when pressure is applied to liquid for filling and a stop for each plunger to assure plunger is not displaced from syringe barrel during filling.

15. A method according to claim 1 wherein said closed sterile Kit comprising step comprises providing a plastic bag for containment of the said sterile closed chamber.

16. A method according to claim 1 wherein said closed sterile Kit comprising step comprises providing a manifold for containment of the said sterile closed chamber.

17. A method for providing a priming cap for a medical syringe needle comprising providing a needle shielding body of said cap, said body comprising a hollow barrel of substantially constant diameter in which a free plunger is disposed, said plunger being pierced by the sharpened end of said needle such that the sharp end of the needle is disposed within a cavity formed by a closed end of said barrel and said plunger such that priming an associated syringe displaces fluid only into said cavity with such fluid being retained in the cavity when said needle is displaced from said cap.

18. A method according to claim 17 wherein a convenience kit made and provided sterile within a plastic bag according to the instant invention comprises providing empty syringes affixed to priming needle caps disposed in a cap-rack within the kit which provides for sterilizing, filling and capping a container within a plastic bag.

19. A method according to claim 1 wherein said particular medicine determining step determines Mitomycin to be the medicine and wherein said kit selecting and acquiring step comprises selecting and providing a kit specifically designed and qualified for compounding and sterilizing Mitomycin medicine and displacing the medicine into at least one pad disposed in a close able cup.

20. A method according to claim 1 wherein said particular medicine determining step determines a hazardous drug to be the medicine and wherein said kit selecting and acquiring step comprises selecting and providing a kit specifically designed and qualified for compounding and sterilizing hazardous drugs and displacing the particular medicine into closed and sealed containers for safety and protection of sterility of the particular medicine.
